(19) United States
(12) Plant Patent Application Publication
(10) Pub. No.: US 2016/0183439 P1
(43) Pub. Date: Jun. 23, 2016

(54) Aloe plant named Aloejaws

(50) ;

(71) Applicant: Johannes Hendrikus Adrianus Ammerlaan, Bleiswijk (NL)

(72) Inventor: Johannes Hendrikus Adrianus Ammerlaan, Bleiswijk (NL)

(73) Assignee: Ovata B.V., Bleiswijk (NL)

(21) Appl. No.: 14/757,065

(22) Filed: Nov. 13, 2015

(30) Foreign Application Priority Data
Dec. 18, 2014 (QZ) 2014/3484

(51) Int. Cl. A01H 5/00

(52) U.S. Cl. PLT/373

(57) ABSTRACT

A new and distinct Aloe cultivar named ‘Aloejaws’ which is characterized by the combination of an upright growth habit, compact size, dark green foliage with an abundance of spines, and the stability of all characteristics from generation to generation. The new variety is a Aloe, typically produced as an indoor ornamental plant.

### CROSS REFERENCE TO RELATED APPLICATIONS

This application claims priority to the Community Plant Variety Rights application number 2014/3484, filed Dec. 18, 2014, which is herein incorporated by reference.

### LATIN NAME OF THE GENUS AND SPECIES

The Latin name of the genus and species of the novel variety disclosed herein is Aloe melanacantha A. Berger.

### VARIETY DENOMINATION

The inventive variety of Aloe disclosed herein has been given the variety denomination ‘Aloejaws’.

### BACKGROUND OF THE INVENTION

Parentage: The Aloe variety ‘Aloejaws’ originated as a naturally occurring, partial-plant mutation of the species Aloe melanacantha (not a named variety or cultivar). The inventor of ‘Aloejaws’ is a commercial ornamental plant producer whom regularly discovers basal shoot mutations of the species Aloe melanacantha at his greenhouse operation in Bleiswijk, The Netherlands. For said mutations which seem to exhibit commercial potential, cuttings are taken to produce trial plants which are subsequently grown for evaluation. Several of such mutations were isolated for evaluation based on darker green coloration of the leaf in combination with other desirable characteristics such as compactness of the plant, upright growth habit, shorter leaves and an abundance of foliar spines. The combination of compactness and upright growth habit results in plants which require less greenhouse bench space for production, less space for shipping and a reduced risk of leaf spines coming into contact with the grower or consumer.

The variety now called ‘Aloejaws’ was initially discovered in 2011 as one such mutation of the parent plant. At the time of discovery, cuttings were taken and the resulting plants were evaluated at the inventor's greenhouse. After further evaluation it was determined that the candidate's dark green foliage, compact size, upright growth habit, shorter leaves and abundance of white foliar spines would prove favorable for commercial marketability. The new variety was given the breeder denomination ‘Aloejaws’.

Asexual Reproduction: ‘Aloejaws’ was first asexually propagated by leaf tip cuttings in October of 2011 at a greenhouse in Bleiswijk, The Netherlands and has since been vegetatively propagated and mericloned through seven additional generation. Through subsequent generations, the unique features of this cultivar are stable and reproduced true to type.

### SUMMARY OF THE INVENTION

The cultivar ‘Aloejaws’ has not been observed under all possible environmental conditions and the phenotype may vary somewhat with variations in environment such as temperature, day length, and light intensity, without, however, any variance in genotype. The following traits have been repeatedly observed and are determined to be the unique characteristics of ‘Aloejaws’. These characteristics in combination distinguish ‘Aloejaws’ as a new and distinct Aloe cultivar:

1. Aloe ‘Aloejaws’ exhibits dark green foliage; and

2. ‘Aloejaws’ exhibits an upright growth habit; and

3. ‘Aloejaws’ exhibits short leaves and a compact size; and

4. ‘Aloejaws’ exhibits an abundance of white foliar spines.

### BRIEF DESCRIPTION OF THE FIGURES

FIG. 1 illustrates, as nearly true as it is reasonably possible to make the same in color photographs of this type, an exemplary plant of ‘Aloejaws’ grown in a grown in a commercial greenhouse in Bleiswijk, The Netherlands. This plant is approximately 12 months old, shown planted in a 12 cm container.

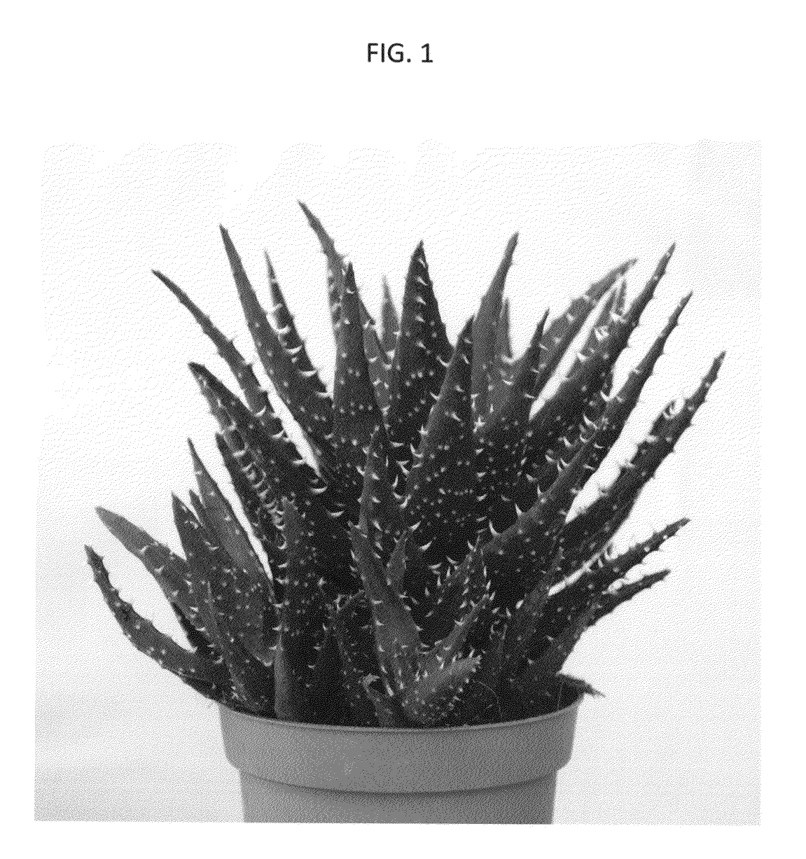

FIG. 2 illustrates, as nearly true as it is reasonably possible to make the same in color photographs of this type, the typical foliage of ‘Aloejaws’.

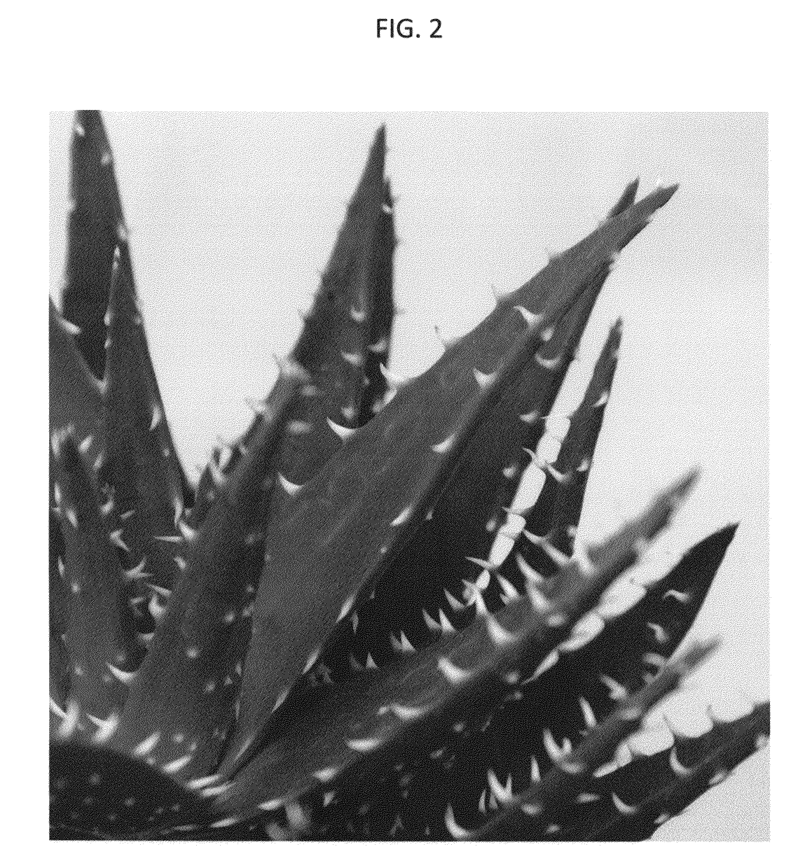

### BOTANICAL DESCRIPTION OF THE PLANT

The following observations and measurements describe a twelve month-old ‘Aloejaws’ plant grown in 12 cm nursery pots at a commercial greenhouse in Bleiswijk, The Netherlands. The plants were grown in full sun to semi-shade. Plants were maintained with a standard fertility program for plants of this type and regularly watered with overhead irrigation as well as through use of ebb-and-flow hydroponic greenhouse benches. No chemical pest measures were taken.

Those skilled in the art will appreciate that certain characteristics will vary with older or, conversely, with younger plants. ‘Aloejaws’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 2015 (sixth edition).

A botanical description of ‘Aloejaws’ and comparisons with other varieties of Aloe are provided below.
Plant description:
   Growth habit.—Broad and upright; overall shape is globular to broad oblong.
   Average height.—16.5 cm, from the soil level to the highest leaf.
   Plant spread.—Average of 19.6 cm.
   Growth rate.—Moderate; approximately 2 cm per month.
   Propagation type.—Vegetative cuttings and mericloning.
   Time to initiate roots.—Approximately 3 weeks to initiate roots at temperatures ranging from 15 to 30 degrees Celsius.
   Time to produce a rooted cutting.—Approximately 6 weeks to produce a rooted cutting.
   Disease resistance.—High disease resistance, similar to the parent.
   Temperature tolerances.—Tolerates temperatures ranging from approximately 12 to 45 degrees Celsius.
Root system:
   General.—Thick; not fibrous.
   Texture.—Slightly fleshy.
   Color.—Greyed-red, near RHS 178A to 178B.
Foliage:
   Arrangement.—Basal rosette. New rosettes are formed at the base of the main rosette, approximately 2 per plant. No stems or lateral branches, the leaves form a stem-like structure not consisting of a stem as such, but only consisting of the leaf sheaths.
   Abundance; density.—Approximately 41 leaves per plant.
   Lamina.—Dimensions — 11.4 cm long (excluding the leaf sheath) and 3.6 cm wide. Thickness — Approximately 0.9 cm. Shape of blade — Lanceolate; slightly carinate. Aspect — Leaves held at an angle of approximately 65 degrees from horizontal. Apex — Acuminate. Base — Sheathing. Margin — Spined. Texture of adaxial surface — Glabrous, moderately glossy. Texture of abaxial surface — Glabrous, slightly glossy; sparsely to moderately covered with soft spines of average length of 1.5 mm and colored white, near RHS NN155D. Color — Juvenile foliage, adaxial surface — Green near RHS 137C. Juvenile foliage, abaxial surface — Green near RHS 137C; spines along the leaf margin are yellow-green, near RHS 145C. Mature foliage, adaxial surface — Green near RHS NN137A. Mature foliage, abaxial surface — Green, in between near RHS 137A and NN137A; spines along the leaf margin are lighter, near RHS 138C to 138D. Venation — No veins are visible.
   Sheath.—Sheath length — Average 2.1 cm. Sheath width — Average 4.0 cm. Sheath color — Adaxial surface is white to orange-white; in between near RHS 155B and 159D, veined greyed-purple RHS 187C. Abaxial surface is white RHS NN155A and veined greyed-yellow near RHS 160A.
Inflorescence: No flowering has been observed to date.

### Comparisons with the Parent Plants

‘Aloejaws’ is similar in many horticultural characteristics to its parent plant, Aloe melanacantha. However ‘Aloejaws’ differs from its parent in the following characteristics:

1. ‘Aloejaws’ exhibits a more upright growth habit when compared to Aloe melanacantha.

2. ‘Aloejaws’ exhibits a smaller plant size when compared to Aloe melanacantha.

3. ‘Aloejaws’ exhibits shorter leaves when compared to the leaves of Aloe melanacantha.

4. The foliage color of ‘Aloejaws’ is dark green, whereas the foliage color of Aloe melanacantha is a lighter green color.

5. The foliage of ‘Aloejaws’ exhibits more spines per leaf when compared to Aloe melanacantha.

That which is claimed is:

1. A new and distinct variety of Aloe plant named ‘Aloejaws’, substantially as described and illustrated herein.
